(19) United States
(12) Patent Application Publication
(10) Pub. No.: US 2020/0104974 A1
(43) Pub. Date: Apr. 2, 2020

(54) SHAPE CONFORMING PROJECTIONS OF MEDICAL INFORMATION

(71) Applicant: Summit Technology Laboratory, Irvine, CA (US)

(72) Inventors: Lohrasb Ross Sayadi, Irvine, CA (US); Mahdi Abbaspour Tehrani, Irvine, CA (US); Alexander Sidenko, Irvine, CA (US)

(21) Appl. No.: 16/584,481

(22) Filed: Sep. 26, 2019

Related U.S. Application Data
(60) Provisional application No. 62/737,680, filed on Sep. 27, 2018.

(51) Int. Cl. G06T 3/00; G06T 11/00; A61B 90/00

(52) U.S. Cl. CPC G06T 3/005; G06T 11/00; A61B 2090/366; G06T 2210/41; A61B 90/36

(57) ABSTRACT

A projection system for projecting medical information onto the surface of the human body such that the medical information conforms and registers to the contour of the underlying shape of the region it is being projected upon. The projection system is in communication with one or more projectors and one or more sensors which capture the topography/area/volume of the region of interest and/or the view of the user, and alter the medical information such that the medical information remains accurate in dimensions when projected into the area of interest.

### CROSS-REFERENCES TO RELATED APPLICATIONS

This application is a non-provisional and claims benefit of U.S. Patent Application No. 62/737,680 filed Sep. 27, 2018, the specification(s) of which is/are incorporated herein in their entirety by reference.

### BACKGROUND OF THE INVENTION

### Field of the Invention

The present invention relates to projection of medical information onto the human body in such a way that the projected information conforms to the shape of the area being projected upon.

### Background Art

In the medical field physicians rely on a whole host of medical information in order to make decisions. For example, surgeons can refer to different images (CT scans, X-rays, MRI's, IR maps etc.) and computer-generated operative plans to help guide decision making in the operating room. However, in its current state, medical information can only be displayed for physicians on either paper, computer screen or through a mixed reality head mounted device such as Holo-Lens. The very primitive paper or computer display which offer visualization of medical information, have a significant limitation in that the operator must mentally manipulate and estimate what he/she sees on the screen to the actual patient. Similarly, in sterile environment such as the operating theatre, paper and computer displays cannot reside in local operative field making the medical information less accessible.

On the other hand, mixed reality technology which uses a head mounted device, display the information to the physician virtually into the operative field solving the issue of accessibility. However, these are personalized systems where the experience is only for the wearer of the head mounted display and have inherent problems such as: alignment/registration issues, the need to wear the technology on the head which may cause user discomfort, limited interaction with the virtual image, and display of information only to the user with the wearable device. Given the issues at hand, a new system is needed to display medical information for medical professionals which does not rely on wearable devices and allows for accurate registration and conformation of medical information onto the patient anatomy.

The present invention features a system that allows for medical information be projected onto the human body while remaining accurate and precise without distortion due to non-flat shape of the underlying part of the human body, objects in the operative field, or movement. Furthermore, the user can interact with the projected information through hand gestures, laser pointers etc. to move and alter the information while maintaining contour conformation.

### BRIEF SUMMARY OF THE INVENTION

In some aspects, the present invention comprises of projection system which is used to project medical information onto the surface of the human body such that the medical information conforms and registers to the contour of the underlying shape of the region it is being projected upon. The projection system is in communication with one or more projectors and one or more sensors (e.g. visible camera, depth camera, IR camera, head/eye tracker) which capture the topography/area/volume of the region of interest and/or the view of the user, and alter the medical information such that the medical information remains accurate in dimensions (length, width, angles, positioning etc.) when projected into the area of interest. The medical information also adapts to the movements of the underlying surface while maintaining shape conformation. This projected information can be altered in real time (moved, repositioned, stretched, lengthened, shortened) by the user while maintaining shape conformation using modalities such as but not limited to hand gestures, markers or digital pens, tablets or other mobile devices. The projected information can change based on the view point of the user. Finally, the projected information can interact with a dye (surface agent) placed on the area being projected upon which marks (i.e. tattoos) the information onto the surface being projected upon.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A-1B show surgical marking (dotted line) for breast reconstruction being projected upon by the present invention. As seen in FIG. 1B, the dotted line surgical markings conform to the region being projected upon (breast) such that the surgical marking bend around the contour of the breast. Without such a system, significant distortion of the medical information may be encountered, altering the accuracy of the surgical markings.

FIG. 2A shows a projection of a checkerboard pattern onto the breast without shape conformation. As seen in the image, the checkerboard pattern is distorted by the contour of the breast causing the angles of each box to be obtuse angles rather than 90 degrees.

FIG. 2B shows a projection of a checkerboard pattern onto the breast with shape conformation. As seen in the image, the checkerboard pattern conforms to the contour which is demonstrated by the ability to maintain the angles of each box at 90 degrees.

FIG. 3 shows an embodiment of the system comprising one or more tiled (to increase coverage or resolution) or superimposed (to increase brightness) projectors, and one or more sensors (e.g. visible camera, depth camera or IR cameras). It also shows the interactive hand or device (e.g. ruler, laser pointer, scalpel).

FIG. 4 shows a non-limiting embodiment of the system that includes a head/eye tracking device and a projection unit.

FIG. 5 shows another non-limiting embodiment of the system that includes multiple ceiling-mounted head/eye tracking devices and a projection unit.

### DETAILED DESCRIPTION OF THE INVENTION

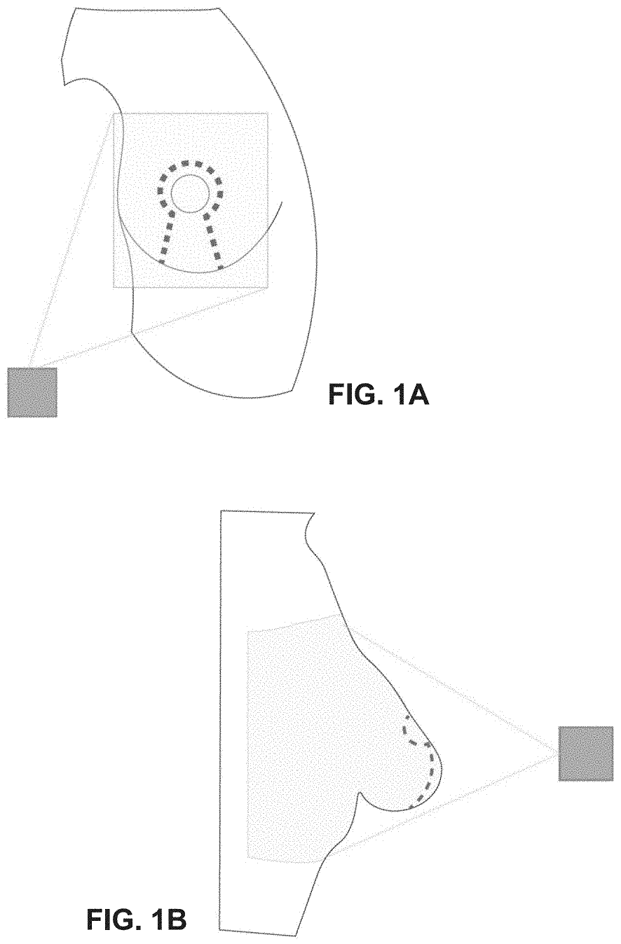

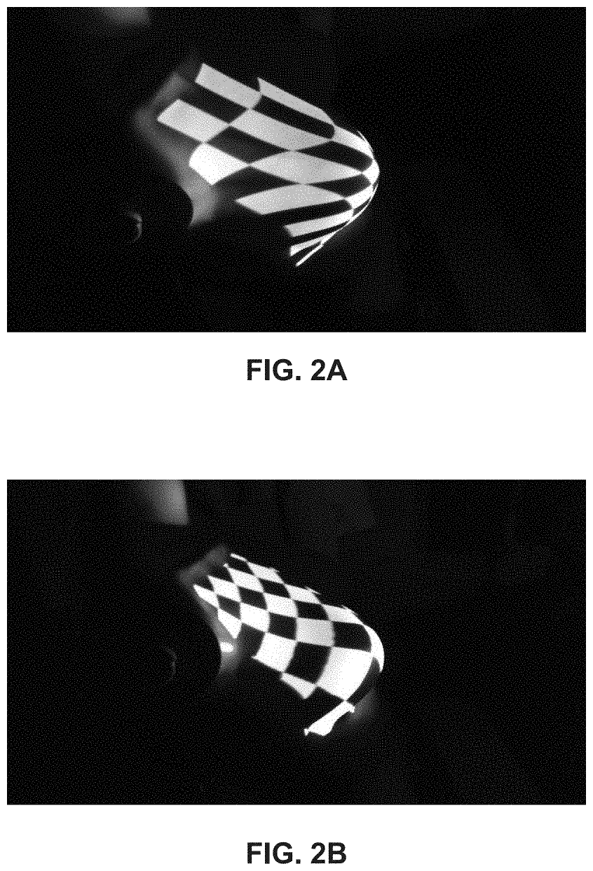

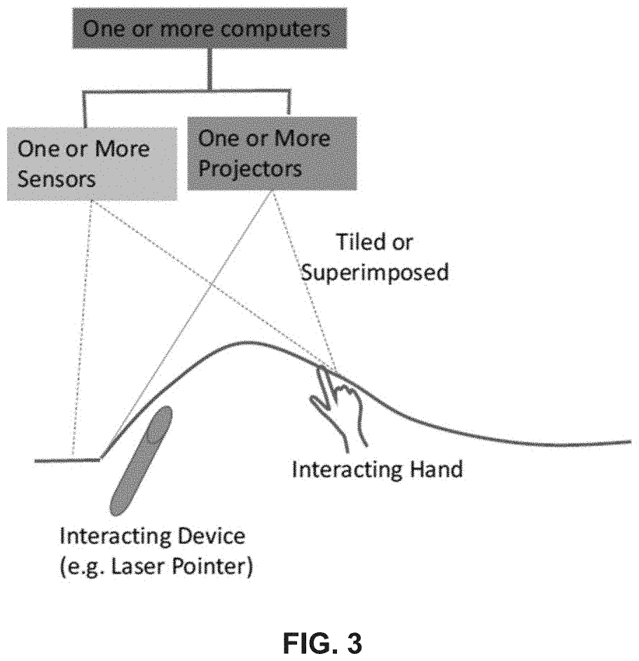

Referring now to FIGS. 1A-5, in some embodiments, the present invention features a system and method of projecting any medical information (i.e. CT scans, X-rays, blood flow map, thermal maps, surgical guides) onto the anatomy (or anatomical models) such that the projection both registers and conforms to the contour of the anatomical region it is being projected upon (i.e. nose, cheeks, breasts, chest, ears etc.), thereby maintaining the accuracy (length, angles positioning) of the medical information.

In another aspect of the present invention, medical information (i.e. CT scans, X-rays, blood flow map, thermal maps, surgical guides) is projected onto the anatomy (or anatomical models) such that the projection adapts to the movement of the surface it is being projected upon (breathing, facial expression) while maintaining conformation to the underlying shape.

In some embodiments, medical information (i.e. CT scans, X-rays, blood flow map, thermal maps, surgical guides) is projected onto the anatomy (or anatomical models) such that the projection can be altered by the user (i.e. stretched, scaled, rotated, repositioned) while maintaining conformation to the underlying shape.

In other embodiments, medical information (i.e. CT scans, X-rays, blood flow map, thermal maps, surgical guides) is projected onto the anatomy (or anatomical models) such that the projection is correct from the single preferred user's (i.e. surgeon) point of view and projection change with the user's head position, in order to correctly visualize internal data (e.g. bones, blood vessels) on the surface.

In some other embodiments, medical information (i.e. CT scans, X-rays, blood flow map, thermal maps, surgical guides) is projected onto the anatomy (or anatomical models) such that the projected information can be adapted to best fit the shape of the area projected upon by integrating contour, shape and volume of the region being projected upon into mathematical consideration.

In additional embodiments of the present invention, projected medical information can be interacted with by the user using via multiple interaction modalities like laser pointers, hand gestures, markers or digital pens, scalpels, tablets or other mobile devices while maintaining conformation to the underlying shape.

In further embodiments of the present invention projected medical information can interact with a surface agent, such as a light activated dye or heat activated dye, placed on the area being projected upon which marks (i.e. Tattoos) the information onto the surface being projected upon.

### Example

The following is a non-limiting example of the present invention. It is to be understood that said example is not intended to limit the present invention in any way. Equivalents or substitutes are within the scope of the present invention.

### Example 1

A surgeon may acquire medical information such as blood flow and vascularity of a patient's breast tissue using and infrared camera or dye-based imaging system and project this medical information on the patient's breast tissue in such a way that the projection conforms to the contour of the breast such that the areas of poor blood flow are accurately demarcated using the projection system. The area of poor blood flow which is projected can be traced using a marker. The traced region can then be surgically removed by the surgeon.

### Example 2

A surgeon may acquire information on the location of major blood vessels in the abdomen using CT scans prior to performing surgery. Intraoperatively the physician can project the preoperative CT scan onto the abdomen in such a way that the projection conforms to the exact contour of the patient's abdomen as well as the view point of the surgeon. Using this technique, the physician can actually identify the location of the major blood vessels for surgical dissection.

### Example 3

Referring to FIGS. 1A-1B, a surgeon may create a computerized patient specific stencil for breast reduction before surgery which they can use during surgery to make their surgical markings. The surgeon can project this stencil on the breast intraoperatively. The computerized template remains accurate (length, angles) while conforming to the area of the breast it is projected upon. The surgeon can alter the projected stencil (stretch, rotate, alter limbs of the stencil) using hand gesture, laser pointer etc. All the while the image shape conforms as it is being altered.

### Example 4

A surgeon preforming craniofacial surgery may project a virtual plan onto the surface of the skull guiding them where to make cuts into the mandible. The projected information registers and conforms to the contour of the mandible maintaining accuracy of the cutting locations that were planned before surgery.

### Example 5

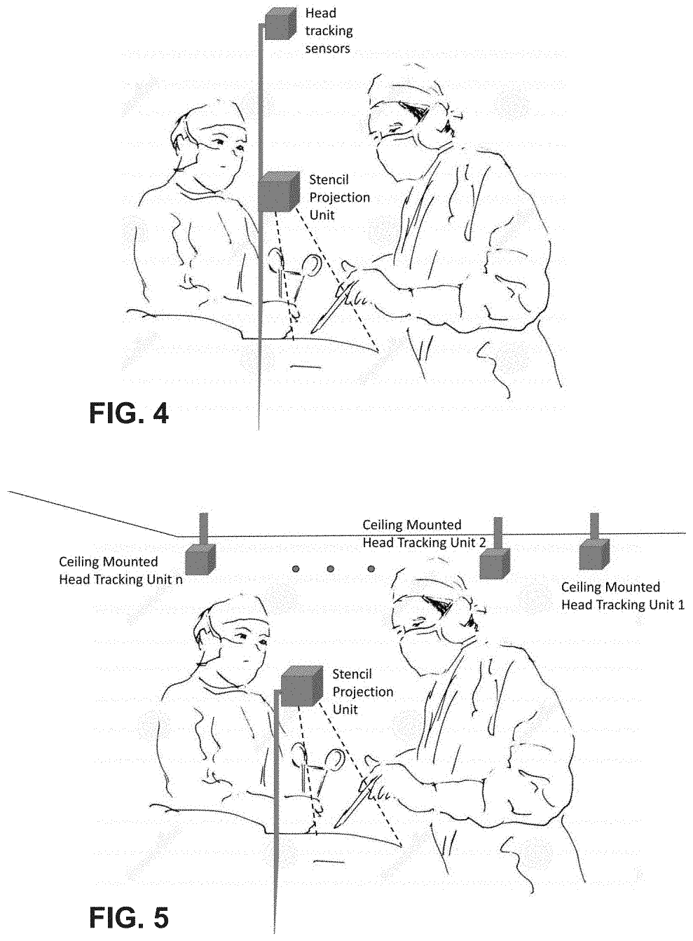

Referring to FIGS. 4-5, a medical student can project an X-ray of the chest onto the chest of an anatomical model such that the projected X-ray registers and conforms to the shape of the chest of the anatomical model and from the student's perspective.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of projecting medical information onto body of a subject such that the information being projected conforms to a shape of a region or anatomy being projected upon while maintaining accuracy of the original shape, wherein:
a. projected information registers or aligns to the region/anatomy being projected upon;
b. projected information is configured to adapt to movement of the projection surface;
c. projected information is altered by the user while maintaining conformation to the underlying shape;
d. projected information is adapted to best fit the shape of the region/anatomy projected upon by integrating contour, shape or volume of the region being projected upon into mathematical consideration; and
e. projected information is configured to be interacted with by the user using via multiple interaction modalities.

2. The method of claim 1, wherein the projected image interacts with a surface agent placed on the area being projected upon which marks the information onto the surface being projected upon.

3. The method of claim 1, wherein the projected information adapts to a movement of a view point of the user.
